(19) United States
(12) Patent Application Publication
(10) Pub. No.: US 2026/0355097 A1
(43) Pub. Date: Nov. 12, 2026

(54) MICROBIOME FRIENDLY SUBSTANCES AND MICROBIOME FRIENDLY COSMETIC FORMULATIONS

(71) Applicant: BASF SE, Ludwigshafen am Rhein (DE)

(72) Inventors: Annette MEHLING, Düsseldorf-Holthausen (DE); Qiu Jing WANG, Hong Kong (CN); Xiao Yue WANG, Shanghai (CN); Qiao Ming ZHANG, Shanghai (CN); Han CAO, Shanghai (CN)

(21) Appl. No.: 19/470,016

(22) PCT Filed: Mar. 25, 2024
(86) PCT No.: PCT/EP2024/057915

§ 371 Date: Sep. 26, 2025

(30) Foreign Application Priority Data
Mar. 29, 2023 (WO) PCT/CN2023/084714

(51) Int. Cl. A61K 8/81; A61K 8/31; A61K 8/34; A61K 8/365; A61K 8/37; A61K 8/46; A61K 8/55; A61K 8/60; A61K 8/73; A61K 8/92; A61Q 19/00

(52) U.S. Cl. CPC A61K 8/8152; A61K 8/31; A61K 8/342; A61K 8/345; A61K 8/365; A61K 8/375; A61K 8/463; A61K 8/553; A61K 8/602; A61K 8/73; A61K 8/922; A61Q 19/00

(57) ABSTRACT

The present invention relates to the use of specific substances as microbiome friendly ingredients in a cosmetic formulation. It furthermore relates to cosmetic formulations having a composition which are microbiome friendly.

The present invention relates to the use of specific substances as microbiome friendly ingredients in a cosmetic formulation. It furthermore relates to cosmetic formulations having a composition which are microbiome friendly.

The present invention relates to the use of formulation ingredients (galenics, e.g. emulsifiers, emollients, rheology modifiers) that do not significantly alter the natural composition of the skin and/or scalp and/or mucous membrane microbial communities and with it the specific microbiomes. As the composition of the microbial communities are adapted to the host and its environment, it plays an essential role in the natural balancing processes which limit growth of pathogens and needed to help maintain the host's health.

The following definition of the skin microbiome essentially refers to a report of the International Cooperation on Cosmetics Regulation dated Jun. 28, 2022. The skin microbiome is present on the whole skin surface, including oral cavity and mucosal surfaces of the external genital organs. The composition of the skin microbiome is dynamic, site-specific but also differs from individual to individual. The skin microbiome includes also microorganisms found on e.g., the scalp, pilosebaceous follicles or sebaceous glands. It also comprises microorganisms detected in the epidermis or dermis. It should be noted that the two terms “microbiota” and “microbiome” are not synonymous as the microbiota only refers to the microorganisms, whereas the microbiome also includes their functional potential. These two terms are mainly used in the scientific literature, whereas in the public domain both terms are often used interchangeably.

The microbiome is a characteristic microbial community occupying a reasonably well-defined habitat which has distinct physio-chemical properties. The microbiome not only refers to the microorganisms involved but also encompasses their theatre of activity, which results in the formation of specific ecological niches. This includes their genetic material, and also structural molecules, like enzymes, membrane lipids or polysaccharides.

The field of microbiome research is one that has made major strides in the past years. Historically, the microorganisms found on or in the human body were generally viewed as being pathogens and as such deleterious to human health. With the introduction of new methodologies, this has proven to be a misconception. The microbial communities on and in our bodies have been found to contribute to our health. The microbes inhabiting e.g. our skin interact both with each other and with their hosts. On the human skin, Staphylococcus epidermis can produce modulins that can hinder the growth of some pathogens; Cutibacterium (formerly Propionibacterium) acnes can degrade triglycerides found in sebum, which in turn sets free fatty acids needed to maintain the natural skin pH and the skin barrier. This further augment the natural immunity of the skin by both inhibiting growth of certain bacteria and by e.g. stimulating the expression of beta defensin 2 by the skin. This intricate interplay is further influenced by various factors, e.g. gender, age, body location, individual susceptibility and the environment. The composition e.g. of the skin's microbiota is adapted to its host and environment. Dysbiosis can lead to skin damage and is associated with skin conditions, such as atopic dermatitis where the pathogen Staphylococcus aureus is involved.

The effects of topical applications on the e.g. the skin's microbiota are increasingly being explored. The use of topically applied substances has the potential of affecting the microbial communities, which in turn can lead to dysbiosis. Currently, the focus is on how bioactives, such as pre-, pro- or postbiotics, can change the microbial composition of the skin. Yet, it is also clear that the healthy balance of the natural microbial inhabitants is also of the essence.

US 2008/0070986 discloses the use of alkyl ether citrates for protecting and maintaining the natural skin/mucous membrane microflora and the microbial skin/mucous membrane ecosystem without impairing the natural balancing processes which limit the number of pathogenic bacteria so that they do not become disease-inducing.

Specchemonline.com, November/December 2021, pages 24-26 disclose microbiome-friendly formulation chassis. Single cosmetic ingredients as well as formulations have been tested in vitro (MIC-tests; MIC: minimal inhibitory concentration) and in vivo.

EP 3 970 802 discloses a skin care composition for promoting the skin microbiome comprising (a) pyruvate and/or lactate in combination with either (b1) urea and/or arginine, or with (b2) caprylic/capric triglycerides, or with (b1) and (b2).

The problem underlying the present invention is to provide ingredients for cosmetic formulations that avoid significantly changing the healthy microbial communities and microbiomes. Such ingredients are hereinafter called microbiome friendly. Formulations which, as a whole, avoid significantly changing the healthy microbial communities and microbiomes are also called microbiome friendly.

If the said ingredients are, as such, known in the state of the art, but their use as a microbiome friendly ingredient in a cosmetic formulation is not known in the state of the art, then the solution of the problem underlying the present invention is this use.

If microbiome friendliness is determined via MIC values, then the MIC value determined for the microorganism Staphylococcus epidermidis when tested in the vehicle ethanol/water (1:1) is especially relevant.

Therefore, the solutions to the problem underlying the present invention, and a first subject of the present invention, is the use of a substance as a microbiome friendly ingredient in a cosmetic formulation, wherein this substance is selected from the group consisting of Ceteareth-12, Cetearyl Alcohol (and) Lecithin (and) Sodium Cetearyl Sulfate (and) Olus Oil, Cetearyl Glucoside (and) Cetearyl Alcohol, Disodium Cetearyl Sulfosuccinate, ethyl linoleate, Glyceryl Oleate, Glyceryl Stearate (and) PEG-100 Stearate, Glyceryl Stearate Citrate, Lauryl Glucoside (and) Polyglyceryl-2 Dipolyhydroxystearate (and) Glycerin, Lecithin, Polyglyceryl-2 Dipolyhydroxystearate, Potassium Cetyl Phosphate, Sodium Cetearyl Sulfate, Laureth-7 Citrate, Glycerin, Dehydroacetic Acid (and) Benzyl Alcohol, Glycerin (and) Aqua (and) Sodium Levulinate (and) Sodium Anisate, Phenoxyethanol (and) Ethylhexylglycerin, Phenoxyethanol (and) Hydroxyacetophenone (and) Caprylyl Glycol (and) Water, Sodium Benzoate, Acrylates/Beheneth-25 Methacrylate Copolymer, Alginate, Caesalpinia spinosa Gum, Glucomannan, Sodium Polyacrylate, Xanthan Gum, 1,4 Butandiol, Butylene Glycol, polyethylene glycol, Triethyl Citrate, Conjugated Linoleic Acid, Vegetable Oil, Passiflora incarnata Seed Oil, Dicaprylyl Carbonate, Isononyl Isononanoate, Coco-Caprylate/Caprate, Dicaprylyl Ether, Caprylyl Caprylate/Caprate, Butyrospermum Parkii (Shea) Butter, Propylheptyl Caprylate, Undecane (and) Tridecane, Citric Acid, Hydrogenated Vegetable Glycerides, Sucrose Polystearate (and) Cetyl Palmitate, Sodium Stearoyl Glutamate, Octyldodecanol, Phenoxyethanol (and) Methylparaben, Polyglyceryl-3 Diisostearate, Cetearyl Alcohol, Ethylparaben (and) Propylene Glycol, Dipropylheptyl Carbonate, Glycerin (and) Myristyl Myristate (and) Ceteareth-12 (and) Glyceryl Stearate (and) Cetearyl Alcohol (and) Potassium Cetyl Phosphate, Glycerin (and) Cetyl Palmitate (and) Ceteareth-12 (and) Glyceryl Stearate (and) Pentylene Glycol (and) Sodium Stearoyl Glutamate (and) Ceteareth-20 (and) Tetrahydroxypropyl Ethylenediamine (and) Cetearyl Alcohol (and) Water, Cetyl Ethylhexanoate, Ethylhexyl Palmitate, Triethylhexanoin, Cocoglycerides, Polyglyceryl-10 stearate, Polyglyceryl-10 Oleate, and Cetearyl Glucoside (and) Cetearyl Alcohol.

One embodiment of the present invention is the use of a substance as a microbiome friendly ingredient in a cosmetic formulation, wherein this substance is an emulsifier. This means that one embodiment of the present invention is the use of a substance as a microbiome friendly ingredient in a cosmetic formulation, wherein this substance is selected from the group consisting of Ceteareth-12, Cetearyl Alcohol (and) Lecithin (and) Sodium Cetearyl Sulfate (and) Olus Oil, Cetearyl Glucoside (and) Cetearyl Alcohol, Disodium Cetearyl Sulfosuccinate, ethyl linoleate, Glyceryl Oleate, Glyceryl Stearate (and) PEG-100 Stearate, Glyceryl Stearate Citrate, Lauryl Glucoside (and) Polyglyceryl-2 Dipolyhydroxystearate (and) Glycerin, Lecithin, Polyglyceryl-2 Dipolyhydroxystearate, Potassium Cetyl Phosphate, Sodium Cetearyl Sulfate, Laureth-7 Citrate, Polyglyceryl-10 stearate, Polyglyceryl-10 Oleate, and Cetearyl Glucoside (and) Cetearyl Alcohol.

A preferred embodiment of the first subject of the present invention is obtained, when the use according to the previous paragraph is restricted to emulsifiers having a MIC value of not below 1. The relevance of the MIC value is explained in the experimental section of the present document. A MIC value of not below 1 essentially means that the substances having such a high MIC value have an especially favorable effect on the microbiome.

Therefore, a preferred embodiment of the first subject of the present invention is the use of a substance as a microbiome friendly ingredient in a cosmetic formulation, wherein this substance is selected from the group consisting of Cetearyl Alcohol (and) Lecithin (and) Sodium Cetearyl Sulfate (and) Olus Oil, Cetearyl Glucoside (and) Cetearyl Alcohol, ethyl linoleate, Glyceryl Oleate, Glyceryl Stearate (and) PEG-100 Stearate, Glyceryl Stearate Citrate, Lecithin, Polyglyceryl-2 Dipolyhydroxystearate and Potassium Cetyl Phosphate.

A more preferred embodiment is obtained, when the use is restricted to emulsifiers having a MIC value of not below 3.

Therefore, a more preferred embodiment of the first subject of the present invention is the use of a substance as a microbiome friendly ingredient in a cosmetic formulation, wherein this substance is selected from the group consisting of Glyceryl Stearate Citrate and Lecithin.

Preferably, the use described in the previous paragraphs is a non-therapeutic use.

A second subject of the present invention corresponds to the use as described in the previous paragraphs. It is a method for cosmetically treating skin in a microbiome friendly way comprising contacting the skin with a substance as defined in the previous paragraphs.

I. e., a second subject of the present invention is a method for cosmetically treating skin in a microbiome friendly way comprising contacting the skin with a substance selected from the group consisting of Ceteareth-12, Cetearyl Alcohol (and) Lecithin (and) Sodium Cetearyl Sulfate (and) Olus Oil, Cetearyl Glucoside (and) Cetearyl Alcohol, Disodium Cetearyl Sulfosuccinate, ethyl linoleate, Glyceryl Oleate, Glyceryl Stearate (and) PEG-100 Stearate, Glyceryl Stearate Citrate, Lauryl Glucoside (and) Polyglyceryl-2 Dipolyhydroxystearate (and) Glycerin, Lecithin, Polyglyceryl-2 Dipolyhydroxystearate, Potassium Cetyl Phosphate, Sodium Cetearyl Sulfate, Glycerin, Dehydroacetic Acid (and) Benzyl Alcohol, Glycerin (and) Aqua (and) Sodium Levulinate (and) Sodium Anisate, Phenoxyethanol (and) Ethylhexylglycerin, Phenoxyethanol (and) Hydroxyacetophenone (and) Caprylyl Glycol (and) Water, Sodium Benzoate, Acrylates/Beheneth-25 Methacrylate Copolymer, Alginate, Caesalpinia spinosa Gum, Glucomannan, Sodium Polyacrylate, Xanthan Gum, 1,4 Butandiol, Butylene Glycol, polyethylene glycol, Triethyl Citrate, Laureth-7 Citrate, Conjugated Linoleic Acid, Vegetable Oil, Passiflora incarnata Seed Oil, Dicaprylyl Carbonate, Isononyl Isononanoate, Coco-Caprylate/Caprate, Dicaprylyl Ether, Caprylyl Caprylate/Caprate, Butyrospermum Parkii (Shea) Butter, Propylheptyl Caprylate, Undecane (and) Tridecane, Citric Acid, Hydrogenated Vegetable Glycerides, Sucrose Polystearate (and) Cetyl Palmitate, Sodium Stearoyl Glutamate, Octyldodecanol, Phenoxyethanol (and) Methylparaben, Polyglyceryl-3 Diisostearate, Cetearyl Alcohol, Ethylparaben (and) Propylene Glycol, Dipropylheptyl Carbonate, Glycerin (and) Myristyl Myristate (and) Ceteareth-12 (and) Glyceryl Stearate (and) Cetearyl Alcohol (and) Potassium Cetyl Phosphate, Glycerin (and) Cetyl Palmitate (and) Ceteareth-12 (and) Glyceryl Stearate (and) Pentylene Glycol (and) Sodium Stearoyl Glutamate (and) Ceteareth-20 (and) Tetrahydroxypropyl Ethylenediamine (and) Cetearyl Alcohol (and) Water, Cetyl Ethylhexanoate, Ethylhexyl Palmitate, Triethylhexanoin, Cocoglycerides, Polyglyceryl-10 stearate, Polyglyceryl-10 Oleate, and Cetearyl Glucoside (and) Cetearyl Alcohol.

Preferred embodiments of the method described in the previous paragraph are methods related to selected substances corresponding to the preferred uses described in this document.

A third subject of the present invention is a substance for use in a method for treatment of the human or animal body by therapy, including prophylaxis, wherein this substance is selected from the group consisting of Ceteareth-12, Cetearyl Alcohol (and) Lecithin (and) Sodium Cetearyl Sulfate (and) Olus Oil, Cetearyl Glucoside (and) Cetearyl Alcohol, Disodium Cetearyl Sulfosuccinate, ethyl linoleate, Glyceryl Oleate, Glyceryl Stearate (and) PEG-100 Stearate, Glyceryl Stearate Citrate, Lauryl Glucoside (and) Polyglyceryl-2 Dipolyhydroxystearate (and) Glycerin, Lecithin, Polyglyceryl-2 Dipolyhydroxystearate, Potassium Cetyl Phosphate, Sodium Cetearyl Sulfate, Glycerin, Dehydroacetic Acid (and) Benzyl Alcohol, Glycerin (and) Aqua (and) Sodium Levulinate (and) Sodium Anisate, Phenoxyethanol (and) Ethylhexylglycerin, Phenoxyethanol (and) Hydroxyacetophenone (and) Caprylyl Glycol (and) Water, Sodium Benzoate, Acrylates/Beheneth-25 Methacrylate Copolymer, Alginate, Caesalpinia spinosa Gum, Glucomannan, Sodium Polyacrylate, Xanthan Gum, 1,4 Butandiol, Butylene Glycol, polyethylene glycol, Triethyl Citrate, Laureth-7 Citrate, Conjugated Linoleic Acid, Vegetable Oil, Passiflora incarnata Seed Oil, Dicaprylyl Carbonate, Isononyl Isononanoate, Coco-Caprylate/Caprate, Dicaprylyl Ether, Caprylyl Caprylate/Caprate, Butyrospermum Parkii (Shea) Butter, Propylheptyl Caprylate, Undecane (and) Tridecane, Citric Acid, Hydrogenated Vegetable Glycerides, Sucrose Polystearate (and) Cetyl Palmitate, Sodium Stearoyl Glutamate, Octyldodecanol, Phenoxyethanol (and) Methylparaben, Polyglyceryl-3 Diisostearate, Cetearyl Alcohol, Ethylparaben (and) Propylene Glycol, Dipropylheptyl Carbonate, Glycerin (and) Myristyl Myristate (and) Ceteareth-12 (and) Glyceryl Stearate (and) Cetearyl Alcohol (and) Potassium Cetyl Phosphate, Glycerin (and) Cetyl Palmitate (and) Ceteareth-12 (and) Glyceryl Stearate (and) Pentylene Glycol (and) Sodium Stearoyl Glutamate (and) Ceteareth-20 (and) Tetrahydroxypropyl Ethylenediamine (and) Cetearyl Alcohol (and) Water, Cetyl Ethylhexanoate, Ethylhexyl Palmitate, Triethylhexanoin, Cocoglycerides, Polyglyceryl-10 stearate, Polyglyceryl-10 Oleate, and Cetearyl Glucoside (and) Cetearyl Alcohol.

A fourth subject of the present invention is a substance for use in a method for treating or preventing a disease caused by a disorder of the skin microbiome, wherein this substance is selected from the group consisting of Ceteareth-12, Cetearyl Alcohol (and) Lecithin (and) Sodium Cetearyl Sulfate (and) Olus Oil, Cetearyl Glucoside (and) Cetearyl Alcohol, Disodium Cetearyl Sulfosuccinate, ethyl linoleate, Glyceryl Oleate, Glyceryl Stearate (and) PEG-100 Stearate, Glyceryl Citrate, Stearate Lauryl Glucoside (and) Polyglyceryl-2 Dipolyhydroxystearate (and) Glycerin, Lecithin, Polyglyceryl-2 Dipolyhydroxystearate, Potassium Cetyl Phosphate, Sodium Cetearyl Sulfate, Glycerin, Dehydroacetic Acid (and) Benzyl Alcohol, Glycerin (and) Aqua (and) Sodium Levulinate (and) Sodium Anisate, Phenoxyethanol (and) Ethylhexylglycerin, Phenoxyethanol (and) Hydroxyacetophenone (and) Caprylyl Glycol (and) Water, Sodium Benzoate, Acrylates/Beheneth-25 Methacrylate Copolymer, Alginate, Caesalpinia spinosa Gum, Glucomannan, Sodium Polyacrylate, Xanthan Gum, 1,4 Butandiol, Butylene Glycol, polyethylene glycol, Triethyl Citrate, Laureth-7 Citrate, Conjugated Linoleic Acid, Vegetable Oil, Passiflora incarnata Seed Oil, Dicaprylyl Carbonate, Isononyl Isononanoate, Coco-Caprylate/Caprate, Dicaprylyl Ether, Caprylyl Caprylate/Caprate, Butyrospermum Parkii (Shea) Butter, Propylheptyl Caprylate, Undecane (and) Tridecane, Citric Acid, Hydrogenated Vegetable Glycerides, Sucrose Polystearate (and) Cetyl Palmitate, Sodium Stearoyl Glutamate, Octyldodecanol, Phenoxyethanol (and) Methylparaben, Polyglyceryl-3 Diisostearate, Cetearyl Alcohol, Ethylparaben (and) Propylene Glycol, Dipropylheptyl Carbonate, Glycerin (and) Myristyl Myristate (and) Ceteareth-12 (and) Glyceryl Stearate (and) Cetearyl Alcohol (and) Potassium Cetyl Phosphate, Glycerin (and) Cetyl Palmitate (and) Ceteareth-12 (and) Glyceryl Stearate (and) Pentylene Glycol (and) Sodium Stearoyl Glutamate (and) Ceteareth-20 (and) Tetrahydroxypropyl Ethylenediamine (and) Cetearyl Alcohol (and) Water, Cetyl Ethylhexanoate, Ethylhexyl Palmitate, Triethylhexanoin, Cocoglycerides, Polyglyceryl-10 stearate, Polyglyceryl-10 Oleate, and Cetearyl Glucoside (and) Cetearyl Alcohol.

A fifth subject of the present invention is a cosmetic formulation having a composition identical to the composition of any of the following formulations or having a composition deviating from the composition of the following formulations in such a way that the amounts of the components of the cosmetic formulation deviate from the amounts of the components of the following formulations by +−20%, preferably +−10%, more preferably by +−5%,

Formulation 1:

| INCI Name | % by weight |
|---|---|
| Sucrose Polystearate (and) | 1.00 |
| Cetyl Palmitate | |
| Cetearyl Alcohol | 4.00 |
| Coco-Caprylate/Caprate | 7.50 |
| Propylheptyl Caprylate | 2.50 |
| Aqua | Add to 100 |
| Glycerin | 3.00 |
| Xanthan Gum | 1.00 |
| Undecane (and) Tridecane | 2.00 |
| Phenoxyethanol (and) Methylparaben, | 1.00 |
| Ethylparaben (and) Propylene Glycol | |

Formulation 2:

| INCI | % by weight |
|---|---|
| Glycerin | 5.00 |
| Butylene Glycol | 10.00 |
| Xanthan Gum | 2.00 |
| Aqua | 63.95 |
| Sodium Benzoate | 0.25 |
| Glycerin, Aqua, Sodium Levulinate, | 1.00 |
| Sodium Anisate | |
| Polyglyceryl-2 Dipolyhydroxystearate | 1.00 |
| Dipropylheptyl Carbonate | 3.00 |
| Dicaprylyl Carbonate | 3.00 |
| Caprylyl Caprylate/Caprate | 10.00 |
| Laureth-7 Citrate | 0.50 |
| Citric Acid | 0.30 |

Formulation 3:

| INCI | % by weight |
|---|---|
| Cetearyl Alcohol, Lecithin, Sodium | 3.00 |
| Cetearyl Sulfate, Olus Oil [EU], | |
| Cetearyl Alcohol, Lecithin, Sodium | |
| Cetearyl Sulfate, Vegetable Oil [CTFA] | |
| Hydrogenated Vegetable Glycerides | 2.50 |
| Caprylyl Caprylate/Caprate | 6.50 |
| Dicaprylyl Carbonate | 4.00 |
| Aqua | 66.35 |
| Butylene Glycol | 10.00 |
| Sodium Benzoate | 0.25 |
| Glycerin | 5.00 |
| Xanthan Gum | 1.00 |
| Glycerin, Aqua, Sodium Levulinate, | 1.00 |
| Sodium Anisate | |
| Citric Acid | 0.40 |

Formulation 4:

| INCI | % by weight |
|---|---|
| Aqua | Add to 100 |
| SODIUM BENZOATE | 0.25 |
| Butylene Glycol | 10.00 |
| Disodium Cetearyl Sulfosuccinate | 1.00 |
| Glycerin | 5.00 |
| Xanthan Gum | 1.00 |
| Polyglyceryl-3 Diisostearate | 2.00 |
| Cetearyl Alcohol | 2.00 |
| Caprylyl Caprylate/Caprate | 5.00 |
| Dicaprylyl Carbonate | 5.00 |
| Undecane (and) Tridecane | 2.00 |
| CITRIC ACID | 1.67 |
| Glycerin (and) Aqua (and) | 1.00 |
| Sodium Levulinate (and) | |
| Sodium Anisate | |

Formulation 5:

| INCI | % by weight |
|---|---|
| Aqua | Add to 100 |
| SODIUM BENZOATE | 0.25 |
| Butylene Glycol | 10.00 |
| Sodium Stearoyl Glutamate | 0.50 |
| Glycerin | 5.00 |
| Xanthan Gum | 1.00 |
| Cetearyl Glucoside (and) | 4.00 |
| Cetearyl Alcohol | |
| Cetearyl Alcohol | 1.00 |
| Caprylyl Caprylate/Caprate | 5.00 |
| Dicaprylyl Carbonate | 5.00 |
| Undecane (and) Tridecane | 2.00 |
| CITRIC ACID | 1.57 |
| Glycerin (and) Aqua (and) | 1.00 |
| Sodium Levulinate (and) | |
| Sodium Anisate | |

Formulation 6:

| INCI | % by weight |
|---|---|
| Aqua | Add to 100 |
| Phenoxyethanol, Ethylhexylglycerin | 1.00 |
| Butylene Glycol | 5.00 |
| Lauryl Glucoside, Polyglyceryl-2 | 3.50 |
| Dipolyhydroxystearate, Glycerin | |
| Cetearyl Alcohol | 1.00 |
| Dicaprylyl Carbonate | 4.00 |
| Butyrospermum Parkii (Shea) Butter | 5.00 |
| Sodium Polyacrylate | 1.50 |

Formulation 7:

| INCI | % by weight |
|---|---|
| Polyglyceryl-10 stearate | 5.00 |
| Cetearyl Alcohol | 3.00 |
| Dicaprylyl Carbonate | 5.00 |
| Coco-Caprylate/Caprate | 5.00 |
| Caprylic/Capric Triglyceride | 3.00 |
| Glycerin | 5.00 |
| Aqua | to 100 |
| Sodium Benzoate | 0.50 |
| Xanthan Gum | 50.00 |
| Glycerin (and) Myristyl Myristate | 10.00 |
| (and) Ceteareth-12 (and) Glyceryl | |
| Stearate (and) Cetearyl Alcohol | |
| (and) Potassium Cetyl Phosphate | |
| Citric Acid | 1.73 |

Formulation 8:

| INCI | % by weight |
|---|---|
| Polyglyceryl-10 Oleate | 5.00 |
| Glycerin | 4.00 |
| Coco-Caprylate/Caprate | 7.00 |
| Aqua | to 100 |
| Phenoxyethanol, Ethylhexylglycerin | 1.00 |
| Glycerin (and) Cetyl Palmitate (and) | 10.00 |
| Ceteareth-12 (and) Glyceryl Stearate | |
| (and) Pentylene Glycol (and) Sodium | |
| Stearoyl Glutamate (and) Ceteareth-20 | |
| (and) Tetrahydroxypropyl Ethylenediamine | |
| (and) Cetearyl Alcohol (and) Water | |
| Citric Acid | q.s. |

Formulation 9:

| INCI | % by weight |
|---|---|
| Caprylyl Caprylate/Caprate | 25.00 |
| Cetyl Ethylhexanoate | 25.00 |
| Ethylhexyl Palmitate | 25.00 |
| Triethylhexanoin | 25.00 |

A further embodiment of the present invention is the non-therapeutic, cosmetic use of any of the formulations 1 to 9 as a microbiome friendly cosmetic formulation.

A further embodiment of the present invention is a method for cosmetically treating skin in a microbiome friendly way comprising contacting the skin with any of the formulations 1 to 9.

A further embodiment of the present invention is a formulation, selected from the group consisting of formulations 1 to 9, for use in a method for treatment of the human or animal body by therapy, including prophylaxis.

A further embodiment of the present invention is a formulation, selected from the group consisting of formulations 1 to 9, for use in a method for treating or preventing a disease caused by a disorder of the skin microbiome.

As the composition of the healthy skin microbiota varies between individuals, the main intent was to identify ingredients that do not disrupt the complex microbial communities found on healthy skin. A selection of ingredients was screened using minimal inhibitory concentration tests (MICs) and/or using 16S rDNA/rRNA microbiome analyses of skin swabs taken before and after 28 days of use by human volunteers with healthy skin (in vivo). Based on the results, skin care formulations were developed and tested in vivo as proof-of-concept studies.

Significantly changing the healthy microbial communities and microbiomes can have several negative consequences. Some of these consequences are merely of cosmetic nature, e. g. red skin, dry skin or the like. Avoiding these consequences therefore is a cosmetic activity and is not therapy or prophylaxis in a medical sense. Other consequences can be pathological and can result in diseases caused by a disorder of the skin microbiome.

The expression “a composition deviating from the composition of a given formulation in such a way that the amounts of the components of the composition deviate from the amounts of the components of the given formulation by +−10%” means that, based on a given formulation having, e. g., 5% by weight Glycerin and 10% Butylene Glycol, any composition having 4.5 to 5.5% by weight Glycerin and 9 to 11% by weight Butylene Glycol is a composition according to the definition given by the said expression.

### EXAMPLES

% means % by weight unless defined differently.

### Test Methods

Hydrophilic ingredients, including emulsifiers and rheology modifiers were evaluated using minimal inhibitory concentration tests (MIC tests). Lipophilic substances, including emollients, and formulations were tested on the skin of human volunteers (in vivo tests).

### Minimal Inhibitory Concentration Tests (MICs)

Minimal Inhibitory Concentration tests are tests typically used to measure growth inhibition by a substance. MICs are defined as the lowest test concentrations of the test substance at which growth of specific microorganisms are inhibited.

In the present case predefined dilutions of the test substance in the appropriate culture medium were prepared and inoculated with the microorganism of interest. After incubation, the test organisms were cultured over a time-period and under conditions normally allowing growth of the microorganism. Vehicle controls (vehicle=solvent used, e. g. water) were also assessed to determine growth without the test substance. The organisms of interest tested were the representative strains S. aureus (found on the skin, often a pathogen), S. epidermidis and Malassezia furfur. The higher the MIC value, the less inhibition of growth is observed. In the following results the test concentrations are given as percent of active substance content.

### In Vivo Tests on Human Skin

In vivo studies were conducted. The general procedure was as follows. At least 20 volunteers having given informed consent and presenting with healthy skin were asked to apply the test sample to the volar forearm twice daily for 28 days. Volunteers were asked to refrain from using topical products 7 days prior to the start and throughout the study. Each test sample was applied randomized to one of four predefined tests area. An untreated area served as a reference. The skin was treated under standardized conditions. The area of interest was swabbed prior to sample application and after 28 days of use. The swabs were stored on dry ice until subjected to genetic analyses.

### Genetic Analyses

Primers targeting variable regions of bacterial 16S rDNA/rRNA were used to amplify the genetic material obtained during swabbing via polymerase chain reaction (PCR) techniques. Following sequencing, the sequences were clustered into operational taxonomic units (OTUs) a 97% similarity threshold defining taxonomic levels, e.g. phyla and genus. Biodiversity was determined using alpha diversity as a measure of microbial richness and abundance and calculating the Shannon diversity index. Wilcoxon signed-rank tests were used to determine statistical significance.

### Results of MIC Test (MIC in % by Weight)

| | | | | | | highest | lowest | |
|---|---|---|---|---|---|---|---|---|
| | | | | | | concentration | concentration | |
| | | | Staphylococcus | Staphylococcus | Malassezia | tested | tested | lowest |
| INCI | Type | pH | aureus | epidermidis | furfur | [AS %] | [AS %] | MIC |
| Ceteareth-12 | Emulsifier | 6.5 | >0.75 | 0.75 | 0.50 | 1.00 | 0.0001 | 0.50 |
| Cetearyl Alcohol | Emulsifier | 5.5 | 1.00 | >3 | 1.00 | 3.00 | 0.0001 | 1.00 |
| (and) Lecithin | | | | | | | | |
| (and) Sodium | | | | | | | | |
| Cetearyl Sulfate | | | | | | | | |
| (and) Olus Oil | | | | | | | | |
| (EU) | | | | | | | | |
| Cetearyl | Emulsifier | 5.5 | 2.00 | 2.00 | | 0.25 | 4.00 | 2.00 |
| Glucoside (and) | | | | | | | | |
| Cetearyl Alcohol | | | | | | | | |
| Disodium | Emulsifier | 5.5 | 0.05 | 0.05 | 1.00 | 3.00 | 0.0001 | 0.05 |
| Cetearyl | | | | | | | | |
| Sulfosuccinate | | | | | | | | |
| ethyl linoleate | Emulsifier | | >1 | >1 | >1 | 1.00 | 0.002 | >1 |
| (not an INCI) | | | | | | | | |
| Glyceryl Oleate | Emulsifier | 5.5 | >2 | >2 | | 2.00 | 0.50 | >2 |
| Glyceryl Stearate | Emulsifier | 5.5 | >2 | >2 | | 2.00 | 0.50 | >2 |
| (and) PEG-100 | | | | | | | | |
| Stearate | | | | | | | | |
| Glyceryl Stearate | Emulsifier | 5.5 | >3 | >3 | >3 | 3.00 | 0.00 | >3 |
| Citrate | | | | | | | | |
| Lauryl Glucoside | Emulsifier | 6.5 | 0.25 | 0.05 | 0.50 | 1.00 | 0.0001 | 0.05 |
| (and) | | | | | | | | |
| Polyglyceryl-2 | | | | | | | | |
| Dipolyhydroxystearate | | | | | | | | |
| (and) Glycerin | | | | | | | | |
| Lecithin | Emulsifier | 5.5 | >3 | >3 | >3 | 3.00 | 0.0001 | >3 |
| Polyglyceryl-2 | Emulsifier | 5.5 | 1.00 | 1.00 | | 4.00 | 0.25 | 1.00 |
| Dipolyhydroxystearate | | | | | | | | |
| Potassium Cetyl | Emulsifier | 5.5 | 1.00 | 0.50 | 1.00 | 3.00 | 0.0001 | 1.00 |
| Phosphate | | | | | | | | |
| Sodium Cetearyl | Emulsifier | 5.5 | 0.08 | 0.50 | >3 | 3.00 | 0.0001 | 0.08 |
| Sulfate | | | | | | | | |
| Laureth-7 Citrate | Emulsifier | 5.5 | 0.75 | 0.75 | 0.75 | 1.00 | 0.0001 | 0.75 |
| Glycerin | Humectant | 5.5 | 20.00 | 30.00 | | 30.00 | 5.00 | 20.00 |
| Dehydroacetic | Preservative | 5.5 | 0.25 | 0.25 | | 0.10 | 2.00 | 0.25 |
| Acid (and) | | | | | | | | |
| Benzyl Alcohol | | | | | | | | |
| Glycerin. Aqua. | Preservative | 5.5 | 2.00 | 2.00 | | 4.00 | 0.25 | 2.00 |
| Sodium | | | | | | | | |
| Levulinate. | | | | | | | | |
| Sodium Anisate | | | | | | | | |
| Phenoxyethanol | Preservative | 5.5 | 0.50 | 0.50 | | 2.00 | 0.10 | 0.50 |
| (and) | | | | | | | | |
| Ethylhexylglycerin | | | | | | | | |
| Phenoxyethanol | Preservative | 5.5 | 1.00 | 1.00 | | 4.00 | 0.25 | 1.00 |
| (and) | | | | | | | | |
| Hydroxyacetophenone | | | | | | | | |
| (and) | | | | | | | | |
| Caprylyl Glycol | | | | | | | | |
| (and) Water | | | | | | | | |
| Sodium Benzoate | Preservative | 5.5 | 0.25 | 0.25 | | 0.10 | 2.00 | 0.25 |
| Acrylates/Beheneth- | Rheology | 5.5 | >1 | >1 | | 1.00 | 0.10 | 1.00 |
| 25 Methacrylate | modifier | | | | | | | |
| Copolymer | | | | | | | | |
| Alginate | Rheology | 5.5 | >2 | >2 | | 2.00 | 0.25 | >2 |
| | modifier | | | | | | | |
| Caesalpinia | Rheology | 5.5 | >2 | >2 | | 2.00 | 0.25 | >2 |
| Spinosa Gum | modifier | | | | | | | |
| Glucomannan | Rheology | 5.5 | >1 | 1.00 | | 1.00 | 0.10 | 1.00 |
| | modifier | | | | | | | |
| Sodium | Rheology | 5.5 | 1.00 | >1 | | 1.00 | 0.10 | 1.00 |
| Polyacrylate | modifier | | | | | | | |
| Xanthan Gum | Rheology | 5.5 | >2 | >2 | | 2.00 | 0.10 | >2 |
| | modifier | | | | | | | |
| 1.4 Butandiol | Solubilizer | 6 | >1 | >1 | >1 | | | >1 |
| Butylene Glycol | Solubilizer | 5.5 | 20.00 | 20.00 | | 5.00 | 30.00 | 20.00 |
| polyethylene | Solubilizer | 5.5 | >2 | >2 | | | | >2 |
| glycol | | | | | | | | |
| Triethyl Citrate | Solubilizer | 5.5 | 1 | 1 | | 2.00 | 0.50 | 1 |
| Conjugated | | | >2 | >2 | >1 | 1.00 | 0.002 | >1 |
| Linoleic Acid (not | | | | | | | | |
| INCI) | | | | | | | | |

These results are to be interpreted as follows. For each substance the lowest MIC value obtained determines whether this substance in useful as microbiome friendly cosmetic ingredient. A substance is useful as microbiome friendly cosmetic ingredient, if this lowest MIC is not lower than a threshold value. This threshold value depends on the type of the substance (solubilizer, emulsifier etc.) and is related to the amount in which these types of substances are generally used in common cosmetic formulations.

For emulsifiers the lowest MIC should not be below 0.05%, preferably not below 0.08%, more preferably not below 0.5%, more preferably not below 1%, more preferably not below 2%, more preferably not below 3%.

For humectants the lowest MIC should not be below 0.1%, preferably not below 0.25%, preferably not below 0.5%, preferably not below 1%, preferably not below 2%.

For preservatives the lowest MIC should not be below 0.25%, preferably not below 0.5%, more preferably not below 1%, more preferably not below 2%.

For rheology modifiers the lowest MIC should not be below 0.1%, preferably not below 0.25%, preferably not below 0.5%, preferably not below 1%, preferably not below 2%.

For solubilizers the lowest MIC should not be below 1%, preferably not below 2%, more preferably not below 20%.

For conjugated linoleic acid the lowest MIC should not be below 1%.

### Results of Genetic Analyses/In Vivo Tests

In vivo tests were carried out with the formulations listed in the following table. Each formulation and the ingredients they contain, was found to be useful as microbiome friendly formulations as well as ingredients. Therefore, any ingredient of the formulations tested can be included in the list of ingredients present in patent claims 1, 3, 4 and 5 of the present patent application as filed.

| | | | % by | | Approx. |
|---|---|---|---|---|---|
| Formulation Nr. | Ingredients | INCI | weight | Function | pH |
| SC-CN-22- | 1.3- | Butylene Glycol | 10.00 | Humectant | 5 |
| VZ011102 | Butanediol | | | | |
| SC-CN-22- | 1.3- | Butylene Glycol | 10.00 | Humectant | 5 |
| VZ011302 | Butanediol | | | | |
| SC-CN-22- | 1.3- | Butylene Glycol | 5.00 | Humectant | 6.00 |
| VZ021001 | Butanediol | | | | |
| SC-DE-20-044-2 | 1.3- | Butylene Glycol | 10.00 | Humectant | 5 |
| | Butanediol | | | | |
| SC-DE-20-067-8 | 1.3- | Butylene Glycol | 10.00 | Humectant | 5 |
| | Butanediol | | | | |
| Cegesoft ® PS 6 + | Cegesoft ® PS 6 | Vegetable Oil | 33.3 | Emollient | n.a. |
| Myritol ® 331 + | | | | | |
| Cetiol ® C 5C | | | | | |
| Cegosoft ® PFO + | Cegosoft ® | Passiflora Incarnata | 33.3 | Emollient | n.a. |
| Eutanol ® G + | PFO | Seed Oil | | | |
| Cetiol ® Ultimate | | | | | |
| SC-DE-20-044-2 | Cetiol ® 4 All | Dipropylheptyl | 3.00 | Emollient | 5 |
| | | Carbonate | | | |
| SC-CN-22- | Cetiol ® CC | Dicaprylyl Carbonate | 5.00 | Emollient | 5 |
| VZ011102 | | | | | |
| SC-CN-22- | Cetiol ® CC | Dicaprylyl Carbonate | 5.00 | Emollient | 5 |
| VZ011302 | | | | | |
| SC-CN-22- | Cetiol ® CC | Dicaprylyl Carbonate | 4.00 | Emollient | 6.00 |
| VZ021001 | | | | | |
| SC-DE-20-044-2 | Cetiol ® CC | Dicaprylyl Carbonate | 3.00 | Emollient | 5 |
| SC-DE-20-067-8 | Cetiol ® CC | Dicaprylyl Carbonate | 4.00 | Emollient | 5 |
| Cetiol ® OE + | Cetiol ® | Isononyl | 33.3 | Emollient | n.a. |
| Cetiol ® Sensoft + | ININ-GL | Isononanoate | | | |
| Cetiol ® ININ-GL | | | | | |
| SC-DE-20-089-1 | Cetiol ® LC | Coco- | 7.5 | Emollient | |
| | | Caprylate/Caprate | | | |
| Cetiol ® OE + | Cetiol ® OE | Dicaprylyl Ether | 33.3 | Emollient | n.a. |
| Cetiol ® Sensoft + | | | | | |
| Cetiol ® ININ-GL | | | | | |
| SC-CN-22- | Cetiol ® RLF | Caprylyl | 5.00 | Emollient | 5 |
| VZ011102 | | Caprylate/Caprate | | | |
| SC-CN-22- | Cetiol ® RLF | Caprylyl | 5.00 | Emollient | 5 |
| VZ011302 | | Caprylate/Caprate | | | |
| SC-DE-20-044-2 | Cetiol ® RLF | Caprylyl | 10.00 | Emollient | 5 |
| | | Caprylate/Caprate | | | |
| SC-DE-20-067-8 | Cetiol ® RLF | Caprylyl | 6.5 | Emollient | 5 |
| | | Caprylate/Caprate | | | |
| SC-CN-22- | Cetiol ® SB 45 | Butyrospermum | 5.00 | Emollient | 6.00 |
| VZ021001 | | Parkii (Shea) Butter | | | |
| SC-DE-20-089-1 | Cetiol ® | Propylheptyl | 2.5 | Emollient | |
| | Sensoft | Caprylate | | | |
| Cetiol ® OE + | Cetiol ® | Propylheptyl | 33.3 | Emollient | n.a. |
| Cetiol ® Sensoft + | Sensoft | Caprylate | | | |
| Cetiol ® ININ-GL | | | | | |
| Cegosoft ® PFO + | Cetiol ® | Undecane (and) | 33.3 | Emollient | n.a. |
| Eutanol ® G + | Ultimate | Tridecane | | | |
| Cetiol ® Ultimate | | | | | |
| SC-CN-22- | Cetiol ® | Undecane (and) | 2.00 | Emollient | 5 |
| VZ011102 | Ultimate | Tridecane | | | |
| SC-CN-22- | Cetiol ® | Undecane (and) | 2.00 | Emollient | 5 |
| VZ011302 | Ultimate | Tridecane | | | |
| SC-DE-20-089-1 | Cetiol ® | Undecane (and) | 2.00 | Emollient | |
| | Ultimate | Tridecane | | | |
| Cegesoft ® PS 6 + | Cetiol ® C 5C | Coco- | 33.3 | Emollient | n.a. |
| Myritol ® 331 + | | Caprylate/Caprate | | | |
| Cetiol ® C 5C | | | | | |
| SC-DE-20-067-8 | Citric Acid | Citric Acid | 0.40 | pH | 5 |
| | (20% | | | Adjustment | |
| | solution) | | | | |
| SC-DE-20-044-2 | Citric Acid | Citric Acid | 0.30 | pH | 5 |
| | (50% | | | Adjustment | |
| | solution) | | | | |
| SC-CN-22- | CITRIC ACID | CITRIC ACID | 1.57 | pH | 5 |
| VZ011102 | 20% | | | Adjustment | |
| SC-CN-22- | CITRIC ACID | CITRIC ACID | | pH | 5 |
| VZ011302 | 20% | | | Adjustment | |
| SC-CN-22- | Cosmedia ® | Sodium Polyacrylate | 1.5 | Rheology | 6.00 |
| VZ021001 | SP | | | modifier | |
| SC-DE-20-067-8 | Cutina ® HVG | Hydrogenated | 2.5 | Consistency | 5 |
| | | Vegetable | | Agent | |
| | | Glycerides | | | |
| SC-DE-20-044-2 | Dehymuls ® | Polyglyceryl-2 | 1.00 | Emulsifier | 5 |
| | PGPH | Dipolyhydroxystearate | | (W/O) | |
| SC-CN-22- | Dermosoft | Glycerin (and) Aqua | 1.00 | Additives | 5 |
| VZ011102 | 1388 Eco | (and) Sodium | | | |
| | | Levulinate (and) | | | |
| | | Sodium Anisate | | | |
| SC-DE-20-044-2 | Dermosoft | Glycerin. Aqua. | 1.00 | Auxiliary | 5 |
| | 1388 eco | Sodium Levulinate. | | | |
| | (Evonik) | Sodium Anisate | | | |
| SC-DE-20-067-8 | Dermosoft | Glycerin. Aqua. | 1.00 | Auxiliary | 5 |
| | 1388 eco | Sodium Levulinate. | | | |
| | (Evonik) | Sodium Anisate | | | |
| SC-CN-22- | Emulgade ® | Cetearyl Glucoside | 4.00 | Emulsifier | 5 |
| VZ011102 | PL 68/50 | (and) Cetearyl | | | |
| | | Alcohol | | | |
| SC-DE-20-089-1 | Emulgade ® | Sucrose | 1.00 | Emulsifier | |
| | Sucro Plus | Polystearate (and) | | | |
| | | Cetyl Palmitate | | | |
| SC-CN-22- | Eumulgin ® | Disodium Cetearyl | 1.00 | Emulsifier | 5 |
| VZ011302 | Prisma | Sulfosuccinate | | | |
| SC-CN-22- | Eumulgin ® | Sodium Stearoyl | 0.50 | Emulsifier | 5 |
| VZ011102 | SG | Glutamate | | | |
| SC-CN-22- | Eumulgin ® VL | Lauryl Glucoside. | 3.5 | Emulsifier | 6.00 |
| VZ021001 | 75 | Polyglyceryl-2 | | | |
| | | Dipolyhydroxystearate. | | | |
| | | Glycerin | | | |
| Cegosoft ® PFO + | Eutanol ® G | Octyldodecanol | 33.3 | Emollient | n.a. |
| Eutanol ® G + | | | | | |
| Cetiol ® Ultimate | | | | | |
| SC-DE-20-089-1 | Euxyl K 320 | Phenoxyethanol (and) | 1.00 | Emollient | |
| | | Methylparaben. | | | |
| SC-CN-22- | Euxyl PE | Phenoxyethanol. | 1.00 | Preservative | 6.00 |
| VZ021001 | 9010 | Ethylhexylglycerin | | | |
| SC-CN-22- | Glycerin | Glycerin | 5.00 | Humectant | 5 |
| VZ011102 | | | | | |
| SC-CN-22- | Glycerin | Glycerin | 5.00 | Humectant | 5 |
| VZ011302 | | | | | |
| SC-DE-20-044-2 | Glycerin | Glycerin | 5.00 | Humectant | 5 |
| SC-DE-20-067-8 | Glycerin | Glycerin | 5.00 | Humectant | 5 |
| SC-DE-20-089-1 | Glycerin | Glycerin | 3.00 | Humectant | |
| SC-CN-22- | Lameform ® | Polyglyceryl-3 | 2.00 | Emulsifier | 5 |
| VZ011302 | TGI | Diisostearate | | | |
| SC-CN-22- | Lanette ® O | Cetearyl Alcohol | 1.00 | Consistency | 5 |
| VZ011102 | | | | agent | |
| SC-CN-22- | Lanette ® O | Cetearyl Alcohol | 2.00 | Consistency | 5 |
| VZ011302 | | | | agent | |
| SC-CN-22- | Lanette ® O | Cetearyl Alcohol | 1.00 | Consistency | 6.00 |
| VZ021001 | | | | agent | |
| SC-DE-20-089-1 | Lanette ® O | Cetearyl Alcohol | 4.00 | Consistency | |
| | | | | agent | |
| Cegesoft ® PS 6 + | Myritol ® 331 | Cocoglycerides | 33.3 | Emollient | n.a. |
| Myritol ® 331 + | | | | | |
| Cetiol ® C 5C | | | | | |
| SC-CN-22- | Na benzoat | SODIUM | 0.25 | Preservative | 5 |
| VZ011102 | | BENZOATE | | | |
| SC-CN-22- | Na benzoat | SODIUM | 0.25 | Preservative | 5 |
| VZ011302 | | BENZOATE | | | |
| SC-DE-20-044-2 | Plantapon ® | Laureth-7 Citrate | 0.50 | Surfactant | 5 |
| | LC 7 | | | | |
| SC-DE-20-067-8 | Plantaquat ® | Cetearyl Alcohol. | 3.00 | Consistency | 5 |
| | NC | Lecithin. Sodium | | agent | |
| | | Cetearyl Sulfate. | | | |
| | | Olus Oil [EU]. | | | |
| | | Cetearyl Alcohol. | | | |
| | | Lecithin. Sodium | | | |
| | | Cetearyl Sulfate. | | | |
| | | Vegetable Oil [CTFA] | | | |
| SC-DE-20-044-2 | Rheocare ® | Xanthan Gum | 2.00 | Stabilizer | 5 |
| | XGN | | | | |
| SC-DE-20-067-8 | Rheocare ® | Xanthan Gum | 1.00 | Rheology | 5 |
| | XGN | | | modifier | |
| SC-DE-20-089-1 | Rheocare ® | Xanthan Gum | 1.00 | Rheology | |
| | XGN | | | modifier | |
| SC-DE-20-044-2 | Sodium | Sodium Benzoate | 0.25 | Preservative | 5 |
| | Benzoate | | | | |
| SC-DE-20-067-8 | Sodium | Sodium Benzoate | 0.25 | Preservative | 5 |
| | Benzoate | | | | |
| SC-CN-22- | Verdessence ™ | Xanthan Gum | 1.00 | Rheology | 5 |
| VZ011102 | Xanthan | | | modifier | |
| SC-CN-22- | Verdessence ™ | Xanthan Gum | 1.00 | Rheology | 5 |
| VZ011302 | Xanthan | | | modifier | |
| SC-CN-22- | Water. demin. | Aqua | Add to 100 | Solvent | 5 |
| VZ011302 | | | | | |
| SC-DE-20-044-2 | Water. demin. | Aqua | 63.95 | | 5 |
| SC-DE-20-067-8 | Water. demin. | Aqua | 66.35 | | 5 |
| SC-DE-20-089-1 | Water. demin. | Aqua | Add to 100 | Solvent | |

### Formulations

The following formulations were used for the genetic analyses/in vivo tests.

Formulation no. SC-DE-20-089-1

| | Trade | INCI | % by | |
|---|---|---|---|---|
| Phase | Name | Name | weight | Function |
| A | Emulgade ® | Sucrose Polystearate (and) | 1.00 | Emulsifier |
| | Sucro Plus | Cetyl Palmitate | | |
| | Lanette ® O | Cetearyl Alcohol | 4.00 | Consistency agent |
| | Cetiol ® LC | Coco-Caprylate/Caprate | 7.50 | Emollient |
| | Cetiol ® | Propylheptyl Caprylate | 2.50 | Emollient |
| | Sensoft | | | |
| B | Water, | Aqua | Add to 100 | Solvent |
| | demin. | | | |
| | Glycerin | Glycerin | 3.00 | Humectant |
| C | Rheocare ® | Xanthan Gum | 1.00 | Rheology modifier |
| | XGN | | | |
| D | Cetiol ® | Undecane (and) Tridecane | 2.00 | Emollient |
| | Ultimate | | | |
| E | Euxyl K 320 | Phenoxyethanol (and) | 1.00 | Preservative |
| | | Methylparaben, | | |
| | | Ethylparaben (and) | | |
| | | Propylene Glycol | | |

Specification

| | |
|---|---|
| pH value (100%; 23 °C) | 6.7 |
| Viscosity (Brookfield; RVT; spindle TE; | 33,750 mPas |
| 4 rpm; with Helipath 23 °C) | |

Formulation no. SC-DE-20-044-2

| | | | % by | |
|---|---|---|---|---|
| Phase | Ingredients | INCI | weight | Function |
| A | Glycerin | Glycerin | 5.00 | Humectant |
| | 1,3- | Butylene Glycol | 10.00 | Humectant |
| | Butanediol | | | |
| | Rheocare ® | Xanthan Gum | 2.00 | Stabilizer |
| | XGN | | | |
| B | Water, | Aqua | 63.95 | |
| | demin. | | | |
| | Sodium | Sodium Benzoate | 0.25 | Preservative |
| | Benzoate | | | |
| | Dermosoft | Glycerin, Aqua, Sodium | 1.00 | Auxiliary |
| | 1388 eco | Levulinate, Sodium Anisate | | |
| | (Evonik) | | | |
| C | Dehymuls ® | Polyglyceryl-2 | 1.00 | Emulsifier (W/O) |
| | PGPH | Dipolyhydroxystearate | | |
| | Cetiol ® 4 All | Dipropylheptyl Carbonate | 3.00 | Emollient |
| | Cetiol ® CC | Dicaprylyl Carbonate | 3.00 | Emollient |
| | Cetiol ® RLF | Caprylyl Caprylate/Caprate | 10.00 | Emollient |
| | Plantapon ® | Laureth-7 Citrate | 0.50 | Surfactant |
| | LC 7 | | | |
| E | Citric Acid | Citric Acid | 0.30 | pH Adjustment |
| | (50% | | | |
| | solution) | | | |

Specification

| | |
|---|---|
| pH value (100%; 23 °C) | 4.9 |
| Viscosity (Brookfield; RVT; spindle TE, | 62,500 mPas |
| Helipath; 4 rpm; 23 °C) | |

Formulation no. SC-DE-20-067-8

| | | | % by | |
|---|---|---|---|---|
| Phase | Ingredients | INCI | weight | Function |
| A | Plantaquat ® | Cetearyl Alcohol, Lecithin, | 3.00 | Consistency |
| | NC | Sodium Cetearyl Sulfate, | | agent |
| | | Olus Oil [EU], Cetearyl | | |
| | | Alcohol, Lecithin, Sodium | | |
| | | Cetearyl Sulfate, | | |
| | | Vegetable Oil [CTFA] | | |
| | Cutina ® | Hydrogenated Vegetable | 2.5 | Consistency |
| | HVG | Glycerides | | Agent |
| | Cetiol ® RLF | Caprylyl Caprylate/Caprate | 6.5 | Emollient |
| | Cetiol ® CC | Dicaprylyl Carbonate | 4.00 | Emollient |
| B | Water, | Aqua | 66.35 | |
| | demin. | | | |
| | 1,3- | Butylene Glycol | 10.00 | Humectant |
| | Butanediol | | | |
| | Sodium | Sodium Benzoate | 0.25 | Preservative |
| | Benzoate | | | |
| C | Glycerin | Glycerin | 5.00 | Humectant |
| | Rheocare ® | Xanthan Gum | 1.00 | Rheology modifier |
| | XGN | | | |
| D | Dermosoft | Glycerin, Aqua, Sodium | 1.00 | Auxiliary |
| | 1388 eco | Levulinate, Sodium Anisate | | |
| | (Evonik) | | | |
| | Citric Acid | Citric Acid | 0.40 | pH Adjustment |
| | (20% | | | |
| | solution) | | | |

Specification

| | |
|---|---|
| pH value (100%; 23 °C) | 4.9 |
| Viscosity (Brookfield; RVT; spindle TE, | 75,000 mPas |
| Helipath; 4 rpm; 23 °C) | |

Formulation no. SC-CN-22-VZ011302

| | | | % by | |
|---|---|---|---|---|
| Phase | Ingredients | INCI | weight | Function |
| A | Water, demin. | Aqua | Add to 100 | Solvent |
| | Na benzoat | SODIUM | 0.25 | Preservative |
| | | BENZOATE | | |
| | 1,3-Butanediol | Butylene Glycol | 10.00 | Humectant |
| | Eumulgin ® | Disodium Cetearyl | 1.00 | Emulsifier |
| | Prisma | Sulfosuccinate | | |
| B | Glycerin | Glycerin | 5.00 | Humectant |
| | Verdessence ™ | Xanthan Gum | 1.00 | Rheology modifier |
| | Xanthan | | | |
| C | Lameform ® | Polyglyceryl-3 | 2.00 | Emulsifier |
| | TGI | Diisostearate | | |
| | Lanette ® O | Cetearyl Alcohol | 2.00 | Consistency agent |
| | Cetiol ® RLF\hat{ } | Caprylyl | 5.00 | Emollient |
| | | Caprylate/Caprate | | |
| | Cetiol ® CC | Dicaprylyl | 5.00 | Emollient |
| | | Carbonate | | |
| D | Cetiol ® | Undecane (and) | 2.00 | Emollient |
| | Ultimate | Tridecane | | |
| E | CITRIC ACID | CITRIC ACID | 1.67 | pH Adjustment |
| | 20% | | | |
| | Dermosoft | Glycerin (and) Aqua | 1.00 | Additives |
| | 1388 Eco | (and) Sodium | | |
| | | Levulinate (and) | | |
| | | Sodium Anisate | | |

Specification

| | |
|---|---|
| pH value (100%; 23 °C) | ~4.95 |
| Viscosity ((Brookfield; DV- I + Viscometer; | ~8,633 mPas |
| spindle 4; 12 rpm; 23 °C) | |

Formulation no. SC-SC-22-VZ011102

| | | | % by | |
|---|---|---|---|---|
| Phase | Ingredients | INCI | weight | Function |
| A | Water, demin. | Aqua | Add to 100 | Solvent |
| | Na benzoat | SODIUM | 0.25 | Preservative |
| | | BENZOATE | | |
| | 1,3-Butanediol | Butylene Glycol | 10.00 | Humectant |
| | Eumulgin ® SG | Sodium Stearoyl | 0.50 | Emulsifier |
| | | Glutamate | | |
| B | Glycerin | Glycerin | 5.00 | Humectant |
| | Verdessence ™ | Xanthan Gum | 1.00 | Rheology modifier |
| | Xanthan | | | |
| C | Emulgade ® PL | Cetearyl Glucoside | 4.00 | Emulsifier |
| | 68/50 | (and) Cetearyl | | |
| | | Alcohol | | |
| | Lanette ® O | Cetearyl Alcohol | 1.00 | Consistency agent |
| | Cetiol ® RLF | Caprylyl | 5.00 | Emollient |
| | | Caprylate/Caprate | | |
| | Cetiol ® CC | Dicaprylyl | 5.00 | Emollient |
| | | Carbonate | | |
| D | Cetiol ® | Undecane (and) | 2.00 | Emollient |
| | Ultimate | Tridecane | | |
| E | CITRIC ACID | CITRIC ACID | 1.57 | pH Adjustment |
| | 20% | | | |
| | Dermosoft | Glycerin (and) Aqua | 1.00 | Additives |
| | 1388 Eco | (and) Sodium | | |
| | | Levulinate (and) | | |
| | | Sodium Anisate | | |

Specification

| | |
|---|---|
| pH value (100%; 23 °C) | ~4.95 |
| Viscosity ((Brookfield; DV- I + Viscometer; | ~13,980 mPas |
| spindle 4; 12 rpm; 23 °C) | |

Formulation no. SC-CN-22-VZ021001

| | | | % by | |
|---|---|---|---|---|
| Phase | Ingredients | INCI | weight | Function |
| A | Water, demin. | Aqua | Add to 100 | Solvent |
| | Euxyl PE 9010 | Phenoxyethanol, | 1.00 | Preservative |
| | | Ethylhexylglycerin | | |
| | 1,3-Butanediol | Butylene Glycol | 5.00 | Humectant |
| B | Eumulgin ® VL | Lauryl Glucoside, | 3.50 | Emulsifier |
| | 75 | Polyglyceryl-2 | | |
| | | Dipolyhydroxystearate, | | |
| | | Glycerin | | |
| | Lanette ® O | Cetearyl Alcohol | 1.00 | Consistency agent |
| | Cetiol ® CC | Dicaprylyl Carbonate | 4.00 | Emollient |
| | Cetiol ® SB 45 | Butyrospermum Parkii | 5.00 | Emollient |
| | | (Shea) Butter | | |
| | Cosmedia ® | Sodium Polyacrylate | 1.50 | Rheology modifier |
| | SP | | | |

Specification

| | |
|---|---|
| pH value (100%; 23 °C) | ~5.97 |
| Viscosity ((Brookfield; DV- I + Viscometer; | ~42,830 mPas |
| spindle 6; 12 rpm; 23 °C) | |

Formulation no. SC-CN-22-VZ092002

| Trade name | INCI | % by weight |
|---|---|---|
| Emulgade ® Verde | Polyglyceryl-10 stearate | 5.00 |
| 10MS | | |
| Lanette ® O | Cetearyl Alcohol | 3.00 |
| Cetiol ® CC | Dicaprylyl Carbonate | 5.00 |
| Cetiol ® C 5C | Coco-Caprylate/Caprate | 5.00 |
| Myritol ® 318 RC | Caprylic/Capric Triglyceride | 3.00 |
| Glycerin | Glycerin | 5.00 |
| Water, demin. | Aqua | to 100 |
| Sodium Benzoate | Sodium Benzoate | 0.50 |
| Rheocare ® XGN | Xanthan Gum | 50.00 |
| (1% solution) | | |
| Mimiskin ™ | Glycerin (and) Myristyl Myristate | 10.00 |
| | (and) Ceteareth-12 (and) Glyceryl | |
| | Stearate (and) Cetearyl Alcohol | |
| | (and) Potassium Cetyl Phosphate | |
| Citric Acid (20%) | Citric Acid | 1.73 |

Formulation no. SC-CN-22-WL092002

| Trade name | INCI | % by weight |
|---|---|---|
| Emulgade ® Verde | Polyglyceryl-10 Oleate | 5.00 |
| 10OL | | |
| Glycerin | Glycerin | 4.00 |
| Cetiol ® C 5C | Coco-Caprylate/Caprate | 7.00 |
| Water, demin. | Aqua | to 100 |
| Euxyl ® PE 9010 | Phenoxyethanol, | 1.00 |
| | Ethylhexylglycerin | |
| Mimiskin ™ Trop | Glycerin (and) Cetyl Palmitate | 10.00 |
| | (and) Ceteareth-12 (and) | |
| | Glyceryl Stearate (and) | |
| | Pentylene Glycol (and) Sodium | |
| | Stearoyl Glutamate (and) | |
| | Ceteareth-20 (and) | |
| | Tetrahydroxypropyl | |
| | Ethylenediamine (and) Cetearyl | |
| | Alcohol (and) Water | |
| Citric Acid Solution | Citric Acid | q.s. |

Formulation no. SC-CN-22-VZ101901

| Trade name | INCI | % by weight |
|---|---|---|
| Cetiol ® RLF | Caprylyl | 25.00 |
| (standard catalyst | Caprylate/Caprate | |
| esterification- | | |
| Non enzymatic | | |
| esterification) | | |
| Cetiol ® SN-1 SD | Cetyl Ethylhexanoate | 25.00 |
| Cegesoft ® C 24 RC | Ethylhexyl Palmitate | 25.00 |
| Myritol ® GTEH-SD | Triethylhexanoin | 25.00 |

1.-9. (canceled)

10. The use of a substance as a microbiome friendly ingredient in a cosmetic formulation, wherein this substance is selected from the group consisting of
Ceteareth-12, Cetearyl Alcohol (and) Lecithin (and) Sodium Cetearyl Sulfate (and) Olus Oil, Cetearyl Glucoside (and) Cetearyl Alcohol, Disodium Cetearyl Sulfosuccinate, ethyl linoleate, Glyceryl Oleate, Glyceryl Stearate (and) PEG-100 Stearate, Glyceryl Stearate Citrate, Lauryl Glucoside (and) Polyglyceryl-2 Dipolyhydroxystearate (and) Glycerin, Lecithin, Polyglyceryl-2 Dipolyhydroxystearate, Potassium Cetyl Phosphate, Sodium Cetearyl Sulfate, Laureth-7 Citrate, Glycerin, Dehydroacetic Acid (and) Benzyl Alcohol, Glycerin (and) Aqua (and) Sodium Levulinate (and) Sodium Anisate, Phenoxyethanol (and) Ethylhexylglycerin, Phenoxyethanol (and) Hydroxyacetophenone (and) Caprylyl Glycol (and) Water, Sodium Benzoate, Acrylates/Beheneth-25 Methacrylate Copolymer, Alginate, Caesalpinia spinosa Gum, Glucomannan, Sodium Polyacrylate, Xanthan Gum, 1,4 Butandiol, Butylene Glycol, polyethylene glycol, Triethyl Citrate, Conjugated Linoleic Acid, Vegetable Oil, Passiflora incarnata Seed Oil, Dicaprylyl Carbonate, Isononyl Isononanoate, Coco-Caprylate/Caprate, Dicaprylyl Ether, Caprylyl Caprylate/Caprate, Butyrospermum Parkii (Shea) Butter, Propylheptyl Caprylate, Undecane (and) Tridecane, Citric Acid, Hydrogenated Vegetable Glycerides, Sucrose Polystearate (and) Cetyl Palmitate, Sodium Stearoyl Glutamate, Octyldodecanol, Phenoxyethanol (and) Methylparaben, Polyglyceryl-3 Diisostearate, Cetearyl Alcohol, Ethylparaben (and) Propylene Glycol, Dipropylheptyl Carbonate, Glycerin (and) Myristyl Myristate (and) Ceteareth-12 (and) Glyceryl Stearate (and) Cetearyl Alcohol (and) Potassium Cetyl Phosphate, Glycerin (and) Cetyl Palmitate (and) Ceteareth-12 (and) Glyceryl Stearate (and) Pentylene Glycol (and) Sodium Stearoyl Glutamate (and) Ceteareth-20 (and) Tetrahydroxypropyl Ethylenediamine (and) Cetearyl Alcohol (and) Water, Cetyl Ethylhexanoate, Ethylhexyl Palmitate, Triethylhexanoin, Cocoglycerides, Polyglyceryl-10 stearate, Polyglyceryl-10 Oleate, and Cetearyl Glucoside (and) Cetearyl Alcohol.

11. The use of a substance as a microbiome friendly ingredient in a cosmetic formulation, according to claim 10, wherein this substance is selected from the group consisting of Ceteareth-12, Cetearyl Alcohol (and) Lecithin (and) Sodium Cetearyl Sulfate (and) Olus Oil, Cetearyl Glucoside (and) Cetearyl Alcohol, Disodium Cetearyl Sulfosuccinate, ethyl linoleate, Glyceryl Oleate, Glyceryl Stearate (and) PEG-100 Stearate, Glyceryl Stearate Citrate, Lauryl Glucoside (and) Polyglyceryl-2 Dipolyhydroxystearate (and) Glycerin, Lecithin, Polyglyceryl-2 Dipolyhydroxystearate, Potassium Cetyl Phosphate, Sodium Cetearyl Sulfate and Laureth-7 Citrate.

12. The use of a substance as a microbiome friendly ingredient in a cosmetic formulation, according to claim 10, wherein this substance is selected from the group consisting of Cetearyl Alcohol (and) Lecithin (and) Sodium Cetearyl Sulfate (and) Olus Oil, Cetearyl Glucoside (and) Cetearyl Alcohol, ethyl linoleate, Glyceryl Oleate, Glyceryl Stearate (and) PEG-100 Stearate, Glyceryl Stearate Citrate, Lecithin, Polyglyceryl-2 Dipolyhydroxystearate and Potassium Cetyl Phosphate.

13. The use of a substance as a microbiome friendly ingredient in a cosmetic formulation, according to claim 10, wherein this substance is selected from the group consisting of Glyceryl Stearate Citrate and Lecithin.

14. A cosmetic formulation having a composition identical to the composition of any of the following formulations or having a composition deviating from the composition of the following formulations in such a way that the amounts of the components of the cosmetic formulation deviate from the amounts of the components of the following formulations by +−20%,
Formulation 1:

| INCI Name | % by weight |
|---|---|
| Sucrose Polystearate (and) Cetyl Palmitate | 1.00 |
| Cetearyl Alcohol | 4.00 |
| Coco-Caprylate/Caprate | 7.50 |
| Propylheptyl Caprylate | 2.50 |
| Aqua | Add to 100 |
| Glycerin | 3.00 |
| Xanthan Gum | 1.00 |
| Undecane (and) Tridecane | 2.00 |
| Phenoxyethanol (and) Methylparaben, | 1.00 |
| Ethylparaben (and) Propylene Glycol | |

Formulation 2:

| INCI | % by weight |
|---|---|
| Glycerin | 5.00 |
| Butylene Glycol | 10.00 |
| Xanthan Gum | 2.00 |
| Aqua | 63.95 |
| Sodium Benzoate | 0.25 |
| Gycerin, Aqua, Sodium Levulinate, | 1.00 |
| Sodium Anisate | |
| Polyglyceryl-2 Dipolyhydroxystearate | 1.00 |
| Dipropylheptyl Carbonate | 3.00 |
| Dicaprylyl Carbonate | 3.00 |
| Caprylyl Caprylate/Caprate | 10.00 |
| Laureth-7 Citrate | 0.50 |
| Citric Acid | 0.30 |

Formulation 3:

| INCI | % by weight |
|---|---|
| Cetearyl Alcohol, Lecithin, Sodium Cetearyl Sulfate, | 3.00 |
| Olus Oil [EU], Cetearyl Alcohol, Lecithin, | |
| Sodium Cetearyl Sulfate, Vegetable Oil [CTFA] | |
| Hydrogenated Vegetable Glycerides | 2.50 |
| Caprylyl Caprylate/Caprate | 6.50 |
| Dicaprylyl Carbonate | 4.00 |
| Aqua | 66.35 |
| Butylene Glycol | 10.00 |
| Sodium Benzoate | 0.25 |
| Glycerin | 5.00 |
| Xanthan Gum | 1.00 |
| Glycerin, Aqua, Sodium Levulinate, Sodium Anisate | 1.00 |
| Citric Acid | 0.40 |

Formulation 4:

| INCI | % by weight |
|---|---|
| Aqua | Add to 100 |
| SODIUM BENZOATE | 0.25 |
| Butylene Glycol | 10.00 |
| Disodium Cetearyl Sulfosuccinate | 1.00 |
| Glycerin | 5.00 |
| Xanthan Gum | 1.00 |
| Polyglyceryl-3 Diisostearate | 2.00 |
| Cetearyl Alcohol | 2.00 |
| Caprylyl Caprylate/Caprate | 5.00 |
| Dicaprylyl Carbonate | 5.00 |
| Undecane (and) Tridecane | 2.00 |
| CITRIC ACID | 1.67 |
| Glycerin (and) Aqua (and) Sodium | 1.00 |
| Levulinate (and) Sodium Anisate | |

Formulation 5:

| INCI | % by weight |
|---|---|
| Aqua | Add to 100 |
| SODIUM BENZOATE | 0.25 |
| Butylene Glycol | 10.00 |
| Sodium Stearoyl Glutamate | 0.50 |
| Glycerin | 5.00 |
| Xanthan Gum | 1.00 |
| Cetearyl Glucoside (and) Cetearyl Alcohol | 4.00 |
| Cetearyl Alcohol | 1.00 |
| Caprylyl Caprylate/Caprate | 5.00 |
| Dicaprylyl Carbonate | 5.00 |
| Undecane (and) Tridecane | 2.00 |
| CITRIC ACID | 1.57 |
| Glycerin (and) Aqua (and) Sodium | 1.00 |
| Levulinate (and) Sodium Anisate | |

Formulation 6:

| INCI | % by weight |
|---|---|
| Aqua | Add to 100 |
| Phenoxyethanol, Ethylhexylglycerin | 1.00 |
| Butylene Glycol | 5.00 |
| Lauryl Glucoside, Polyglyceryl-2 | 3.50 |
| Dipolyhydroxystearate, Glycerin | |
| Cetearyl Alcohol | 1.00 |
| Dicaprylyl Carbonate | 4.00 |
| Butyrospermum Parkii (Shea) Butter | 5.00 |
| Sodium Polyacrylate | 1.50 |

Formulation 7:

| INCI | % by weight |
|---|---|
| Polyglyceryl-10 stearate | 5.00 |
| Cetearyl Alcohol | 3.00 |
| Dicaprylyl Carbonate | 5.00 |
| Coco-Caprylate/Caprate | 5.00 |
| Caprylic/Capric Triglyceride | 3.00 |
| Glycerin | 5.00 |
| Aqua | to 100 |
| Sodium Benzoate | 0.50 |
| Xanthan Gum | 50.00 |
| Glycerin (and) Myristyl Myristate (and) | 10.00 |
| Ceteareth-12 (and) Glyceryl Stearate | |
| (and) Cetearyl Alcohol (and) Potassium | |
| Cetyl Phosphate | |
| Citric Acid | 1.73 |

Formulation 8:

| INCI | % by weight |
|---|---|
| Polyglyceryl-10 Oleate | 5.00 |
| Glycerin | 4.00 |
| Coco-Caprylate/Caprate | 7.00 |
| Aqua | to 100 |
| Phenoxyethanol, Ethylhexylglycerin | 1.00 |
| Glycerin (and) Cetyl Palmitate (and) | 10.00 |
| Ceteareth-12 (and) Glyceryl Stearate | |
| (and) Pentylene Glycol (and) Sodium | |
| Stearoyl Glutamate (and) Ceteareth-20 | |
| (and) Tetrahydroxypropyl Ethylenediamine | |
| (and) Cetearyl Alcohol (and) Water | |
| Citric Acid | q.s. |

Formulation 9:

| INCI | % by weight |
|---|---|
| Caprylyl Caprylate/Caprate | 25.00 |
| Cetyl Ethylhexanoate | 25.00 |
| Ethylhexyl Palmitate | 25.00 |
| Triethylhexanoin | 25.00 |

15. A cosmetic formulation comprising the formulation according to claim 14, wherein the cosmetic formulation is microbiome friendly.

16. A method for cosmetically treating skin in a microbiome friendly way comprising contacting the skin with the formulation according to claim 14.

17. A formulation according to claim 14 for use in a method for treatment of the human or animal body by therapy, including prophylaxis.

18. A formulation according to claim 14 for use in a method for treating or preventing a disease caused by a disorder of the skin microbiome.
